(19) United States
(12) Patent Application Publication
(10) Pub. No.: US 2011/0152989 A1
(43) Pub. Date: Jun. 23, 2011

(54) SOFT ABRASION-RESISTANT POLYISOBUTYLENE URETHANE COPOLYMERS

(75) Inventor: Jiahong Tan, Canyon Country, CA (US)

(73) Assignee: PACESETTER, INC., Sylmar, CA (US)

(21) Appl. No.: 12/646,273

(22) Filed: Dec. 23, 2009

(51) Int. Cl. A61N 1/05; C08G 18/32

(52) U.S. Cl. 607/122; 528/65

(57) ABSTRACT

A polyisobutylene polyurethane (PIBU) copolymer comprising a polyisobutylene (PIB) having a molecular weight of about 400 to about 5,000 daltons; a hard segment (PU) formed from reacting the PIB with diisocyanates and from reacting one of the diisocyanate linked to the PIB with a chain extender. The chain extender has a length based on a number of carbon atoms in the chain extender. A shore hardness of the PIBU copolymer is determined, in part, by either one or more of a PIB:PU ratio, the length of PIB, the type of diisocyanate, and the type and length of the chain extender.

### FIELD OF THE INVENTION

The present disclosure relates to novel copolymers and, more particularly, to polyisobutylene urethane copolymers which may be used in connection with implantable medical devices.

### BACKGROUND

Various types of materials have been used to enhance the biocompatibility and durability of medical devices which are implanted in patients. For example, implantable cardiac leads are implanted in a patient to deliver electrical stimulation to a patient's heart. In addition to concerns regarding biocompatibility of the implanted cardiac leads, there are concerns regarding its durability. After a lead is implanted in a patient, it may be subject to abrasive wear from rubbing against another lead, another implanted device or the patient's anatomical structure. Abrasive wear can eventually cause breaks or tears in the lead body's insulating housing and consequent failure of the electrical connection provided by one or more of the electrical conductors. A short circuit, in particular, can potentially damage the circuits of the implantable medical device to an extent requiring its replacement. Insulation abrasion failures account for the largest proportion of all failures in silicone rubber insulated leads.

It is therefore preferable for implantable leads to have a housing or an outer surface that is resistant to abrasive wear. Various types of materials, such as silicone rubber, polyurethane, and polystyrene-isobutylene-styrene (PIBS) triblock polymers have been used to insulate various medical devices that are implanted in the body. Silicone has been known to have superior flexibility and long term biostability; however, silicone has relatively poor abrasion and tear resistance. Polyurethane, on the other hand, is more resistant to abrasion, cuts and tears, but is more susceptible to biodegradation. In addition, because polyurethane is relatively stiff, it often causes the lead to perforate the heart.

Thus, there continues to be a need for materials for implantable leads that are biostable and flexible, while at the same time having improved resistance to abrasion and tears.

### SUMMARY

In one preferred embodiment, polyisobutylene polyurethane (PIBU) copolymers are described. The PIBU copolymer is synthesized using polyisobutylene (PIB), diisocyanate and chain extender. The PIB has a molecular weight of about 400 to about 5,000 daltons. Excess diisocyanate is reacted with PIB through its end hydroxyl group to form an isocynate-terminated prepolymer. The chain extender has a length based on the number of carbon atoms in the chain extender. At least one end isocyanate group of the prepolymer reacts with the chain extender to form the PIBU. The hard segments (PU) of the PIBU copolymer is formed from the reacting diisocyanates with the PIB and also from the reacting a chain extender with the diisocyanate. In a preferred embodiment, the chain extender is reacted with only one of the diisocyanate that has been reacted with the PIB. There is no particular order required of reacting the diisocyanate with the PIB and reacting the chain extender with the diisocyanate, as the order may be reversed to achieve the same result. Thus, in a preferred embodiment, the PIBU copolymer is flanked on both sides of the PIB segment by the hard segments PU. The desired shore hardness of the PIBU copolymer may be manipulated based on either one or more of a PIB:PU ratio, the length of PIB, the type of diisocyanate, and the type and length of the chain extender. For example, increasing the PIB:PU ratio and/or increasing the length of the PIB will decrease the Shore A hardness of the PIBU copolymer.

In another preferred embodiment, a PIBU copolymer is described as comprising the general formula:

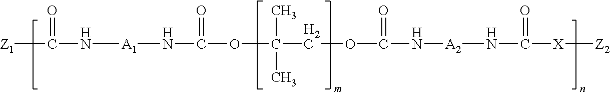

wherein

A₁ and A₂ may be the same or different and is any one or more selected from the group consisting of an alkyl, an alkylene, a cycloalkyl, a cycloalkylene, an aryl, an arylalkyl, an arylakylene, and a polycyclic aryl, and an alkenylaryl group;

X is an —O—R—O— group or an —HN—R—NH— group and wherein R is a linear or branched aliphatic or aromatic group; m is from 5 to 100; and n is from 50 to 800.

In accordance with this preferred embodiment, the soft segment of the PIBU is represented by the PIB structure —(—CH₂—C(CH₃)₂—)ₘ—, and is flanked on both sides by the hard segments PU (the remaining structure within the outer bracketed structure n).

In a further preferred embodiment, a method of producing a PIBU copolymer is described. The method comprises reacting a polyisobutylene diol with a diisocyanate and to produce an isocyanate-terminated prepolymer and reacting the isocyanate-terminated prepolymer with a chain extender comprising one of an aliphatic diol, an aliphatic diamine, an aromatic diol, or an aromatic diamine.

Implantable medical devices, such as cardiac leads, are also described as comprising a layer formed from the PIBU copolymers. In accordance with one aspect of the preferred embodiment, the PIBU copolymers form the insulation layer of a cardiac lead. In accordance with another aspect of the preferred embodiment, the PIBU copolymers form a surrounding layer or sheath of a cardiac lead. In a preferred embodiment, the PIBU copolymer has a Shore A hardness in the range of about 50 A to about 75 A, preferably in the range of about 60 A to about 70 A, and most preferably about 65 A.

A more complete understanding of methods disclosure will be afforded to those skilled in the art, as well as a realization of additional advantages and objects thereof, by a consideration of the following detailed description. Reference will be made to the appended sheets of drawings which will first be described briefly.

### BRIEF DESCRIPTION OF THE DRAWINGS

FIG. 1 is a side view of an implantable cardiac pacing, sensing and cardioverting/defibrillating system, including a lead.

FIG. 2 is a transverse cross-sectional view of the lead as seen along the line 2-2 of FIG. 1.

FIG. 3 is a longitudinal cross-sectional view of the lead tubular body as taken along section line 3-3 in FIG. 2.

Throughout the several figures and in the specification that follows, like element numerals are used to indicate like elements appearing in one or more of the figures.

### DETAILED DESCRIPTION OF THE PREFERRED EMBODIMENTS

The polyisobutylene polyurethane (PIBU) copolymers described herein are particularly suitable for use in connection with implantable medical devices and, more particularly, for use in connection with fabricating abrasion-resistant outer surfaces for implantable cardiac leads.

Various types of abrasion-resistant materials have been used in connection implantable cardiac leads. Pellethane 2363 55D (“Pellethane”), for example, is a benchmark polyurethane that is used for pacing and ICD lead insulation. While it shows good mechanical properties, Pellethane suffers the disadvantage of being stiff. This stiffness, in turn, increases the risk of causing unwanted tissue perforation.

Softer, abrasion-resistant materials have therefore been proposed for use in connection with implantable cardiac leads. One such example is a polystyrene-isobutylene-styrene (“PIBS”) triblock copolymer. While the PIBS copolymers have a much lower hardness than many of the other insulation materials (e.g., silicone, Pellethane), the soft PIBS material often has a sticky and rough surface, making it difficult to handle in the assembly of leads.

Other abrasion-resistant materials used in connection with cardiac leads are described in U.S. Pat. No. 6,990,378, issued Jan. 24, 2006, entitled “Abrasion-Resistant Implantable Medical Lead and a Method of Fabricating Such a Lead”; U.S. patent application Ser. No. 11/671,887, filed Feb. 6, 2007, entitled “Implantable Medical Lead with Insulation Formed of Polystyrene-b-Polyisobutylene-b-Polystyrene (SIBS) Blends” and U.S. patent application Ser. No. 11/681,409, filed Mar. 2, 2007, entitled “Polyurethane/Polystyrene-b-Polyisobutylene-b-Polystyrene Composites for Implantable Medical Device Applications,” the disclosures of each of which are incorporated herein in each of their entireties.

The PIBU copolymers described herein provide an improved abrasion-resistant material for use in connection with cardiac leads. One of the many advantages of the PIBU copolymers is that it is characterized as being relatively soft and flexible, while at the same time being capable of forming a material having a surface that is less rough or sticky as the PIBS. The PIBU copolymers also abrasion-resistant and biostable due to the presence of polyisobutylene (“PIB”). In a preferred embodiment, the PIBU copolymer comprises at least 30% by weight of PIB and, more preferably, at least 50% by weight of PIB.

In accordance with one embodiment, the PIBU copolymer comprises a PIB having a molecular weight of about 400 to about 5,000 daltons, at least two urethane groups (“U”) linked to the PIB and a chain extender having a length based on the number of carbon atoms in the chain extender. A shore hardness of the PIBU copolymer is determined, in part, by either one or more of a PIB:PU ratio, the length of PIB, the type of diisocyanate, and the type and length of the chain extender.

The PIB in the PIBU copolymer has the general formula —(—CH₂—C(CH₃)₂—)ₘ— and the long PIB segments of its polymer chains provide good flex properties. The desired shore hardness of the PIBU copolymer may be manipulated by either increasing or decreasing the PIB:PU ratio. Increasing the PIB:PU ratio also increases the abrasion-resistant properties of the PIBU copolymer. In a preferred embodiment, PIB at least 30%, and more preferably, 50% of the total weight of the PIBU copolymer. In accordance with this preferred embodiment, the total molecular weight of the PIBU copolymer may be in the range from about 15,000 to about 250,000 daltons.

The at least two urethane linkages in the PIBU copolymers may be linked directly or indirectly to the PIB. The urethane linkages are segments consisting of a chain of organic units joined by urethane (carbamate) links and may generally be represented by the formula —O—(CO)NHAₓ-, wherein x is 1 or 2 and A₁ and A₂ may be the same or different and is any one or more selected from the group consisting of an alkyl, an alkylene, a cycloalkyl, a cycloalkylene, an aryl, an arylalkyl, an arylakylene, and a polycyclic aryl, and an alkenylaryl group. The at least two urethane linkages in the PIBU copolymer may be the same or they may be different.

The chain extender has a length based on the number of carbon atoms in the chain extender. In a preferred embodiment, the chain extender may be linked directly or indirectly to the reacted diisocyanate group. In another preferred embodiment, the chain extender may be linked directly to PIB. In a particularly preferred embodiment, the chain extender is either one of an HO—R—OH diol or an H₂N—R—NH₂ diamine, wherein R is a linear or branched aliphatic or aromatic group. The shore hardness of the PIBU copolymer may be decreased by increasing the length of the chain extender.

The PIBU copolymer described in accordance with the present disclosure comprises the general chemical formula:

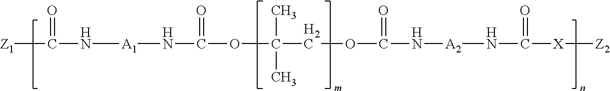

wherein

A₁ and A₂ may be the same or different and is any one or more selected from the group consisting of an alkyl, an alkylene, a cycloalkyl, a cycloalkylene, an aryl, an arylalkyl, an arylakylene, and a polycyclic aryl, and an alkenylaryl group;

X is an —O—R—O— group or an —HN—R—NH— group and wherein R is a linear or branched aliphatic or aromatic group;

m is from 5 to 100, and preferably 7 to 90; and

n is from 50 to 800, and preferably 100-500.

In a preferred embodiment, A₁ and A₂ is selected from the group consisting of: a methylene diphenyl, a polymeric methylene diphenyl, a methylbenzyl, a naphthyl, a hexyl, a methylene bis(p-cyclohexyl), and a dicyclohexylmethyl. In a particularly preferred embodiment, A₁ and A₂ is a methylene diphenyl.

The chain extender is represented by X, which is preferably C₂-C₁₀ linear alkyl group. In accordance with this embodiment, a longer carbon chain provides a softer, more flexible PIBU copolymer. Z₁ and Z₂ represents the terminal groups of the PIBU copolymer. In a preferred embodiment, Z₁ and Z₂ is a C₂-C₄ alkoxide group. Z₁ and Z₂ may be the same or different.

A method of producing a PIBU copolymer is also described herein. The method comprises a two step synthesis in which (1) n mol polyisobutylene diol is reacted with 2 n mol diisocyanates and to produce n mol isocyanate-terminated prepolymer; and (2) the n mol isocyanate-terminated prepolymer is reacted with n mol aliphatic diol, aliphatic diamine, aromatic diol, or aromatic diamine to produce the PIBU copolymer. In accordance with this method, n is any positive integer greater than or equal to 1.

The polyisobutylene diol comprises the general structure:

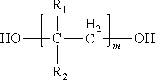

wherein R₁ and R₂ is an aliphatic or an aromatic group and wherein R₁ and R₂ may be the same or different; and

wherein m is from 5 to 100.

In a preferred embodiment, the R₁ and R₂ are —CH₃ group and the diisocyanate is any one or more selected from the group consisting of: a methylene diphenyl diisocyanate, a toluene diisocyanate, a hexamethyldiisocyanate, an isophorone diisocyanate, a naphthalene diisocyanate, a 1,6-hexane diisocyanate, a methylene bis(p-cyclohexyl isocyanate), and a polymeric methylene diisocyanate. In accordance with this preferred embodiment, the polymeric MDI has the general formula:

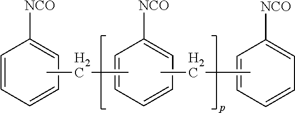

In a particularly preferred embodiment, the diisocyanate is a methylene-diphenyl diisocyanate (“MDI”). MDI exists in three isomers, 2,2′-MDI, 2,4′-MDI and 4-4′-MDI. In accordance with a preferred embodiment, the 4-4′-MDI is reacted with the polyisobutylene diol as follows:

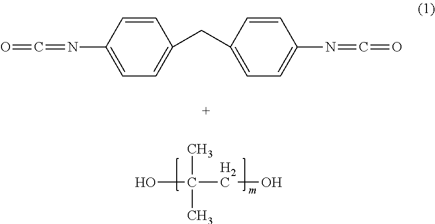

The reaction temperature may be from room temperature to 100° C. and the reaction time may be anywhere from 30 minutes to 10 hours. The ratio diisocyanate to PIB diol may range from 3:1 to 1:1.

The isocyanate terminated prepolymer resulting from the reaction (1) above comprises the general structure:

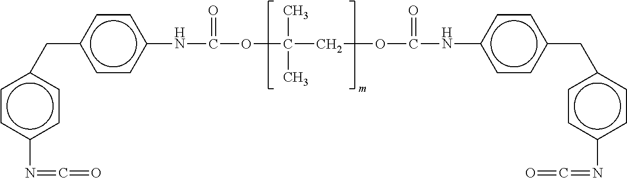

In the second reacting step, the isocyanate terminated prepolymer is reacted with a chain extender. The chain extender may be a linear or branched aliphatic diol or an aromatic diol. The chain extender may also be a linear or branched aliphatic diamine or an aromatic diamine. In a preferred embodiment, the chain extender is a linear C₂-C₁₀ aliphatic diol, preferably a 1,4-butane diol. Thus, the second reaction step is illustrated as follows:

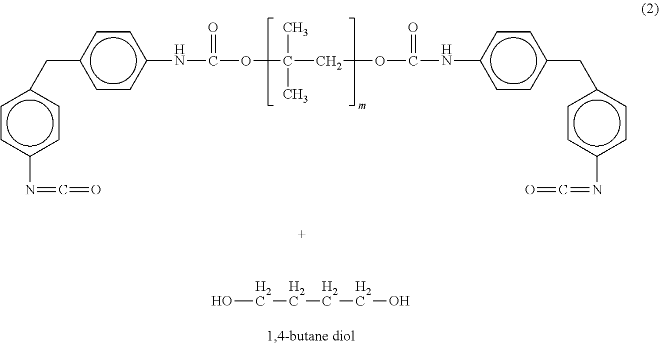

Again, the reaction may proceed in a temperature range of about room temperature to 100° C. and the reaction time may range from 30 minutes to 10 hours. The final total isocyanate/hydroxyl or amine, or hydroxyl plus amine may range from about 1.0 to about 1.1. The resulting polyisobutylene polyurethane from reaction step (2) comprises the general chemical formula:

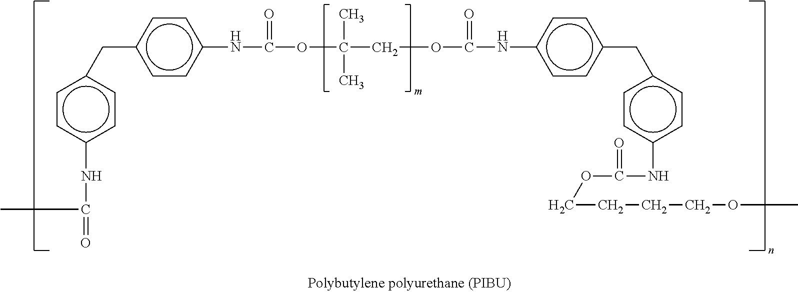

Abrasion-resistant coatings, layers or sheaths have been overlaid over the outer circumferential surfaces of tubular bodies of silicone leads to increase the abrasion resistance of the leads. In accordance with one preferred embodiment, the abrasion-resistant layers or sheaths may be formed from an extruded PIBU copolymer described herein.

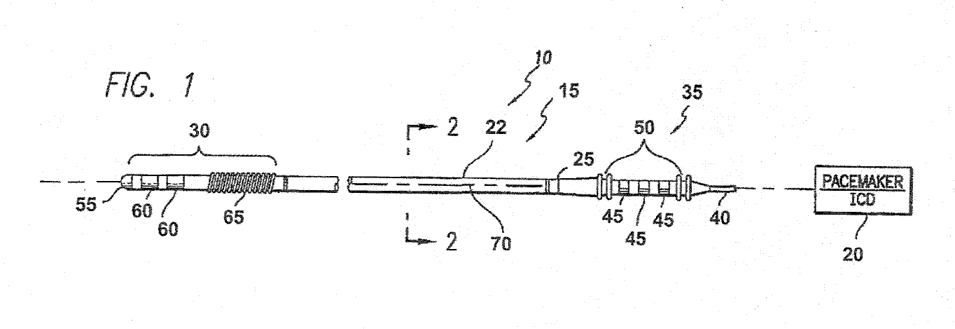

For a discussion of an embodiment of a lead 15 employing the PIBU copolymer insulation 75, reference is made to FIG. 1, which is a side view of a cardiac resynchronization therapy (“CRT”) system 10. As shown in FIG. 1, in one embodiment, the CRT system 10 includes a lead 15 and a pacemaker, a defibrillator or ICD 20. In one embodiment, the lead 15 includes a tubular body having a proximal end 25 and a distal end 30. In one embodiment, the lead 15 is of a quadrupolar design, but in other embodiments the lead 15 will be of a design having a greater or lesser number of poles.

In one embodiment, the lead tubular body 22 has a generally circular or round cross-section. In other embodiments, the lead tubular body 22 has other cross-sections that are generally non-circular or non-round (e.g., elliptical, squared, etc.).

In one embodiment, the lead body 22 may be isodiametric (i.e., the outside diameter of the lead body 22 may be the same throughout its entire length. In one embodiment, the outside diameter of the lead body 22 may range from approximately 0.026 inch (2 French) to about 0.130 inch (10 French).

As depicted in FIG. 1, a connector assembly 35 proximally extends from the proximal end 25 of the lead 15. In one embodiment, the connector assembly 35 is compatible with a standard such as the IS-4 standard for connecting the lead body to the ICD 20. The connector assembly 35 includes a tubular pin terminal contact 40 and ring terminal contacts 45. The connector assembly 22 of the lead 15 is received within a receptacle (not shown) in the ICD 20 containing electrical terminals positioned to engage the contacts 40, 45 on the connector assembly 35. As is well known in the art, to prevent ingress of body fluids into the receptacle, the connector assembly 35 is provided with spaced sets of seals 50. In accordance with standard implantation techniques, a stylet or guide wire (not shown) for delivering and steering the distal end of the lead body during implantation is inserted into a lumen of the lead body 22 through the tubular connector terminal pin 40.

As illustrated in FIG. 1, in one embodiment, the distal end 30 of the lead body 22 carries one or more electrodes 55, 60, 65 having configurations, functions and placements along the length of the distal end 30 dictated by the desired stimulation therapy, the peculiarities of the patient's anatomy, and so forth. The lead body 22 shown in FIG. 1 illustrates but one example of the various combinations of stimulating and/or sensing electrodes 55, 60, 65 that may be utilized.

As depicted in FIG. 1, in one embodiment, the distal end 30 of the lead body 22 includes one tip electrode 55, two ring electrodes 60 and a single cardioverting/defibrillating coil 65. The tip electrode 55 forms the distal termination of the lead body 22. The ring electrodes 60 are just distal of the tip electrode 55. The cardioverter/defibrillator coil 65 is just distal of the ring electrodes 60. Depending on the embodiment, the tip and ring electrodes 55, 60 may serve as tissue-stimulating and/or sensing electrodes.

In other embodiments, other electrode arrangements will be employed. For example, in one embodiment, the electrode arrangement may include additional ring stimulation and/or sensing electrodes 60 as well as additional cardioverting and/or defibrillating coils 65 spaced apart along the distal end of the lead body 22. In one embodiment, the distal end 30 of the lead body 22 may carry only pacing and sensing electrodes, only cardioverting/defibrillating electrodes, or a combination of pacing, sensing and cardioverting/defibrillating electrodes.

The distal end 30 of the lead body 22 may include passive fixation means (not shown) that may take the form of conventional projecting tines for anchoring the lead body within the right atrium or right ventricle of the heart. Alternatively, the passive fixation or anchoring means may comprise one or more preformed humps, spirals, S-shaped bends, or other configurations manufactured into the distal end 30 of the lead body 22 where the lead 15 is intended for left heart placement within a vessel of the coronary sinus region. The fixation means may also comprise an active fixation mechanism such as a helix. It will be evident to those skilled in the art that any combination of the foregoing fixation or anchoring means may be employed.

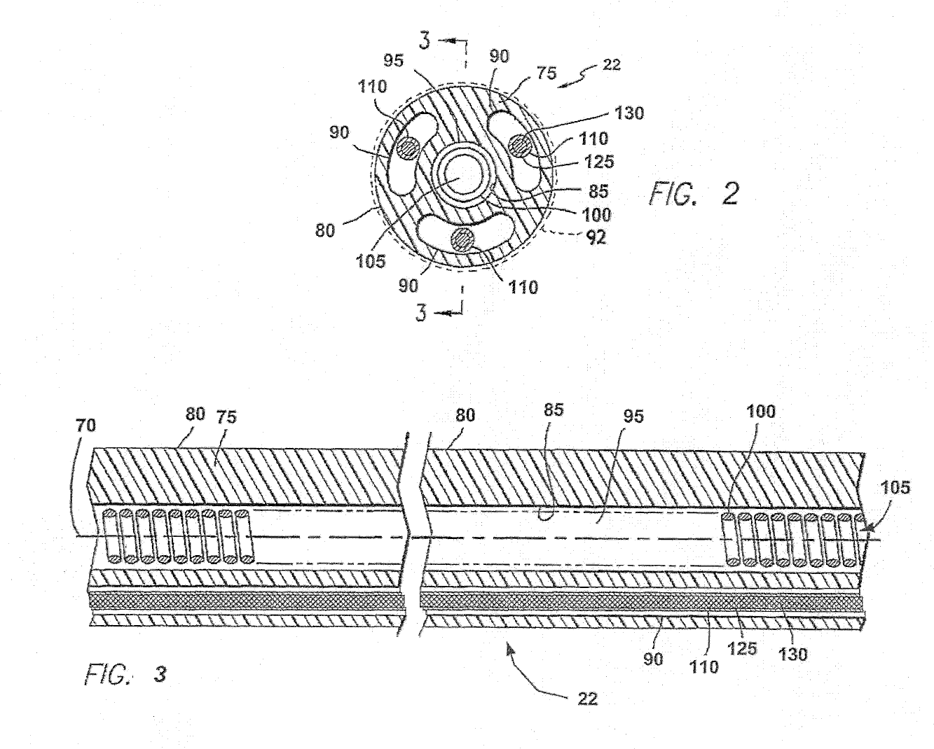

For a discussion regarding the construction of the tubular body 22 of the lead 15, reference is made to FIGS. 1-3. FIG. 2 is a transverse cross-section of the lead tubular body 22 as taken along section line 2-2 in FIG. 1. FIG. 3 is a longitudinal cross-section of the lead tubular body 22 as taken along section line 3-3 in FIG. 2.

As depicted in FIGS. 2 and 3, in one embodiment, the lead 15 includes an insulation wall 75 that has an outer circumferential surface 80, an inner circumferential surface 85 and one or more wall lumens 90. In one embodiment, a wall lumen 90 will have a generally circular or round cross-section. In other embodiments, a wall lumen 90 will have other cross-sections that are generally non-circular or non-round (e.g., arcuate or arched as shown in FIG. 2, elliptical, squared, triangular, etc.). As indicated in FIGS. 1 and 3, the lead body 22 extends along a central longitudinal axis 70. In one embodiment, the insulation layer or wall 75 is made of the PIBU copolymer.

In one embodiment, the inner circumferential surface 85 of the insulation wall 75 defines a central lumen 95. In one embodiment, a helical coil extends through the central lumen 95 and electrically connects the tubular connector terminal pin 40 with the tip electrode 55. The helical coil 100 defines a coil lumen 105 through which a stylet or guide wire can extend during implantation of the lead 15. In other embodiments, the central lumen 95 does not have a helical coil 100 extending through the central lumen 95. Instead, a liner made of a polymer such as PTFE extends through and lines the central lumen 95 to provide a slick or lubricious surface for facilitating the passage of the guide wire or stylet through the central lumen 95.

In another embodiment, each wall lumen 90 may include one or more conductor cables 110 extending through the lumen. In other embodiments wherein the insulation wall 75 does not have any wall lumens 90, the cables will extend through the insulation layer 75 by having the insulation wall 75 co-extruded along the cables 110. The cables or wires 110 may further comprise a polymer insulation layer or jacket 125 and a core 130.

In other embodiments, as indicated by phantom line in FIG. 2, a coating, jacket or sheath (“layer”) 92 extends over the outer circumferential surface 80 of the insulation layer 75. In one embodiment, the insulation wall 75 is silicone rubber, silicone polyurethane copolymer, Pellethane, or a blended SIBS material and the layer 92 is one of the aforementioned PIBU copolymer. Regardless of whether the PIBU copolymer is used to form the insulation wall 75 or a layer 92 extending over the insulation wall 75, the result is a lead 15 employing the PIBU copolymer having increased flexibility and biostability.

In one embodiment, as illustrated in FIG. 2, the insulation wall 75 has three arcuately or radially extending wall lumens 90. In other embodiments, the wall lumen will have other shapes (e.g., square, rectangular, circular, oval, etc.) and/or the insulation wall 75 will have a greater or lesser number of wall lumens 90. In other embodiments, the insulation wall or layer 75 will not have any wall lumens 90.

As indicated in FIGS. 2 and 3, in one embodiment, the outer circumferential surface 80 of the insulation wall 75 forms the overall outer circumferential surface of the lead body 22. In other embodiments, a layer 92 extends over the outer circumferential surface 80 of the insulation wall 75 to a greater or lesser extent. For example, in one embodiment and in accordance with well-known techniques, the outer surface of the lead body 22 may have a lubricious coating along its length to facilitate its movement through a lead delivery introducer and the patient's vascular system.

Having thus described preferred embodiments for PIBU copolymers, methods of producing PIBU copolymers, and implantable medical devices comprising PIBU copolymers, it should be apparent to those skilled in the art that certain advantages of the disclosure have been achieved. It should also be appreciated that various modifications, adaptations, and alternative embodiments thereof may be made without departing from the scope and spirit of the present technology. The following claims define the scope of what is claimed.

What is claimed is:

1. A polyisobutylene polyurethane (PIBU) copolymer comprising:
a polyisobutylene segment (PIB) having a molecular weight of about 400 to about 5,000 daltons;
a hard segment (PU) formed from reacting the PIB with diisocyanates and from reacting a chain extender with at least one of the diisocyanates, the chain extender having a length based on a number of carbon atoms in the chain extender;
wherein a shore hardness of the PIBU copolymer is determined at least by either one or more of a PIB:PU ratio, the length of PIB, the type of diisocyanate, and the type and length of the chain extender.

2. The PIBU copolymer of claim 1, wherein the Shore hardness of the PIBU copolymer is decreased by increasing the PIB:PU ratio.

3. The PIBU copolymer of claim 1, wherein the Shore hardness of the PIBU copolymer is decreased by increasing the length of the PIB.

4. The PIBU copolymer of claim 1, wherein the Shore hardness of the PIBU copolymer is decreased by increasing the length of the chain extender.

5. The PIBU copolymer of claim 1, wherein the PIB comprises at least 50% of the total weight of the PIBU copolymer.

6. The PIBU copolymer of claim 1, wherein the total weight of the PIBU copolymer is from about 15,000 to about 250,000 Daltons.

7. The PIBU copolymer of claim 6, wherein the PIBU has a Shore A hardness in the range of 50 A to 75 A.

8. The PIBU copolymer of claim 1, wherein the chain extender is linked to at least one isocyanate group.

9. The PIBU copolymer of claim 8, wherein the chain extender is either one of an HO—R—OH diol or an H₂N—R—NH₂ diamine, wherein R is a linear or branched aliphatic or aromatic group.

10. An implantable medical device having a layer formed from the PIBU of claim 1.

11. The implantable medical device of claim 10, wherein the device is a lead comprising a surrounding layer that is formed from the PIBU.

12. A PIBU copolymer comprising the general chemical formula: 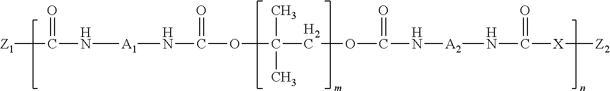
wherein
A₁ and A₂ may be the same or different and is any one or more selected from the group consisting of an alkyl, an alkylene, a cycloalkyl, a cycloalkylene, an aryl, an arylalkyl, an arylakylene, and a polycyclic aryl, and an alkenylaryl group;
X is an —O—R—O— group or an —HN—R—NH— group and wherein R is a linear or branched aliphatic or aromatic group;
m is from 5 to 100; and
n is from 50 to 800.

13. The PIBU copolymer of claim 12, wherein A₁ and A₂ is selected from the group consisting of: a methylene diphenyl, a polymeric methylene diphenyl, a methylbenzyl, a naphthyl, a hexyl, a methylene bis(p-cyclohexyl), and a dicyclohexylmethyl.

14. The PIBU copolymer of claim 13, wherein A₁ and A₂ is a methylene diphenyl.

15. The PIBU copolymer of claim 12, wherein X is an —O—R—O— group and wherein R is a linear C₂-C₁₀ alkyl.

16. The PIBU copolymer of claim 12, wherein X is an —HN—R—NH— group and wherein R is a linear C₂-C₁₀ alkyl.

17. The PIBU copolymer of claim 12, wherein Z₁ and Z₂ is a C₂-C₄ alkoxide group and wherein Z₁ and Z₂ may be the same or different.

18. The PIBU copolymer of claim 12, wherein m is from 7 to 90.

19. The PIBU copolymer of claim 12, wherein n is from 100-500.

20. An implantable medical device having a layer formed from the PIBU of claim 12.

21. The implantable medical device of claim 20, wherein the device is a lead comprising a surrounding layer that is formed from the PIBU.

22. A method of producing a PIBU copolymer comprising:
reacting n mol polyisobutylene diol with 2 n mol diisocyanate and to produce n mol isocyanate-terminated prepolymer; and
reacting the isocyanate-terminated prepolymer with n mol of an aliphatic diol, an aliphatic diamine, an aromatic diol, or an aromatic diamine.

23. The method of claim 22, polyisobutylene diol comprises the general structure: 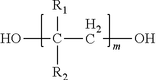
wherein R₁ and R₂ is an aliphatic or an aromatic group and wherein R₁ and R₂ may be the same or different; and
wherein m is from 5 to 100.

24. The method of claim 22, wherein R₁ and R₂ are —CH₃ group.

25. The method of claim 22, wherein the diisocyanate is any one or more selected from the group consisting of: a methylene diphenyl diisocyanate, a toluene diisocyanate, a hexamethyldiisocyanate, an isophorone diisocyanate, a naphthalene diisocyanate, a 1,6-hexane diisocyanate, a methylene bis(p-cyclohexyl isocyanate), and a polymeric methylene diisocyanate.

26. The method of claim 22, wherein the isocyanate terminated prepolymer comprises the general structure: 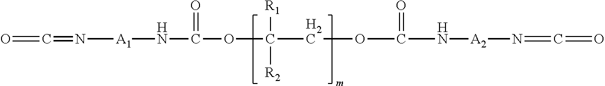
wherein
R₁ and R₂ is an aliphatic or an aromatic group;
A₁ and A₂ is any one or more selected from the group consisting of an alkyl, an alkylene, a cycloalkyl, a cycloalkylene, an aryl, an arylalkyl, an arylakylene, and a polycyclic aryl, and an alkenylaryl group; and
m is from 5 to 100.

27. The method of claim 26, wherein the second reacting step is performed with an aliphatic diol.

28. The method of claim 27, wherein the aliphatic diol is a 1,4-butane diol.
